(19) United States
(12) Patent Application Publication
(10) Pub. No.: US 2010/0331278 A1
(43) Pub. Date: Dec. 30, 2010

(54) USES OF CATIONIC HYDROXYETHYLCELLULOSE IN ORAL INGESTION FORMS, AND PREVENTION AND TREATMENT OF METABOLIC DISORDERS

(76) Inventors: W. H. Kerr Anderson, Midland, MI (US); Shao-Ching Hung, Midland, MI (US); William T. Stott, Traverse City, MI (US); Maciej Turowski, Midland, MI (US); Wallace H. Yokoyama, Berkeley, CA (US); Scott A. Young, Midland, MI (US)

Correspondence Address: The Dow Chemical Company, P.O. BOX 1967, 2040 Dow Center, Midland, MI, 48641, US

(21) Appl. No.: 12/775,180

(22) Filed: May 6, 2010

Related U.S. Application Data
(60) Provisional application No. 61/176,611, filed on May 8, 2009.
(60) Provisional application No. 61/178,162, filed on May 14, 2009.

(51) Int. Cl. A61K 31/717; C08B 11/08; A61P 3/04; A61P 3/00; A61P 3/06; A61P 3/10

(52) U.S. Cl. 514/57; 536/96

(57) ABSTRACT

Described are an oral ingestion form comprising cationic hydroxyethylcellulose, and methods of using the same in prevention and treatment of metabolic disorders.

### CROSS-REFERENCE TO RELATED APPLICATIONS

This application claims the benefit of priority from U.S. Provisional Patent Application Nos. 61/176,611, filed May 8, 2009, and 61/178,162, filed May 14, 2009, which are incorporated by reference herein as if in their entireties.

### STATEMENT REGARDING FEDERALLY SPONSORED RESEARCH

This invention was made under a Cooperative Research And Development Agreement with the US Department of Agriculture, number 58-3K95-5-1072.

### FIELD

This invention relates to an oral ingestion form comprising cationic hydroxyethylcellulose, and methods of using the same in prevention and treatment of metabolic disorders.

### BACKGROUND

Obesity, one of many metabolic disorders, has risks so well known as to need no introduction. In fact, metabolic disorders represent some of the most significant health risks of our time. For example, another metabolic disorder, Type II diabetes (characterized by both impaired insulin secretion and insulin resistance), has increased at such an alarming rate over the past thirty years that if this trend is not reversed, more than 33% individuals born in 2000 are expected to develop Type II diabetes in their lifetime.

Currently, the American Heart Association estimates that about 20 to 25 percent of US adults have “metabolic syndrome,” a metabolic disorder characterized by: abdominal obesity, atherogenic dyslipidemia, hypertension, insulin resistance with or without glucose intolerance, proinflammatory state and prothrombotic state (Grundy et al., “Definition Of Metabolic Syndrome” Circulation, 2004, V109, pages 433-438, Document Number DOI: 10.1161/01.CIR.0000111245.75752.C6). It is generally recognized in the art that people with three or more of the above symptoms can be considered to have metabolic syndrome. People with metabolic syndrome are at increased risk of a cardiovascular disease, such as coronary heart disease or other diseases related to plaque buildups in artery walls (e.g., stroke and peripheral vascular disease) and/or Type II diabetes.

WO 2008/051794 outlines the dangers of metabolic syndrome, as well as certain water-soluble cellulose derivatives that are useful in methods of preventing or treating metabolic syndrome or a symptom or condition associated with the metabolic syndrome in an individual. WO 2008/051794 describes a number of cellulose derivatives, including hydroxyethylcellulose.

However, quaternary ammonium hydroxyethylcellulose, also known as cationic hydroxyethylcellulose (“c-HEC”), is not disclosed in WO 2008/051794. Moreover, c-HEC (unlike hydroxyethylcellulose) is not addressed in the United States Food and Drug Administration's CFR, Title 21. c-HEC is not addressed in the European Union's E number system for approved additives, either. Thus, one skilled in the art would not consider the use of c-HEC to be suggested by disclosure of hydroxyethylcellulose in WO 2008/051794. Arguably, in fact, c-HEC cannot currently be considered a dietary cellulose derivative or other dietary fiber.

The art continues to recognize a need for preventing or treating metabolic disorders. By examining key biomarkers, like serum cholesterol, insulin, glucose, leptin, and adiponectin, and features like body and organ weight, the present invention demonstrates the efficacy of c-HEC for preventing or treating metabolic disorders.

### SUMMARY

In one embodiment, the present invention provides an oral ingestion form comprising cationic hydroxyethylcellulose.

In one embodiment, the present invention provides a method of preventing or treating a metabolic disorder in an individual, comprising administering to the individual an effective amount of a water-soluble cationic hydroxyethylcellulose.

### DETAILED DESCRIPTION

In one embodiment, the present invention provides an oral ingestion form comprising cationic hydroxyethylcellulose. “Oral ingestion form” refers to any conventional means for an individual to ingest a solid, gel, or liquid, including, but not limited to medicaments, foods, beverages, food additives, nutraceuticals, or dietary supplements. “Ingest” is to take internally, as for digestion.

Although WO 2001/048021 mentions cationic celluloses might be useful for pharmaceutical controlled release, e.g., buccal drug delivery (drug administration through the mucosal membranes lining the cheeks), Applicants believe that ingestion of cationic hydroxyethylcellulose, such as in an oral ingestion form, is not known to those skilled in the art, and in fact would be discouraged by them (for example, due to the presence of glyoxal, or postulated mucoadhesion properties).

As a matter of background, cellulose is a linear, unbranched polysaccharide composed of anhydroglucose monosaccharide units linked through their 1,4 positions by the β anomeric configuration. Substitution of the hydroxyl groups (with positions at 2, 3, or 6) will yield cellulose derivatives. The theoretical limit of hydroxyl substitution is three. As not every anhydroglucose unit will be substituted identically, the average number of hydroxyl groups substituted per anhydroglucose unit is referred to as the degree of substitution, i.e., as a mean over the whole polymer chain. For purposes of this specification, “cationic hydroxyethylcellulose” refers to cellulose derivatives having a Formula (I):

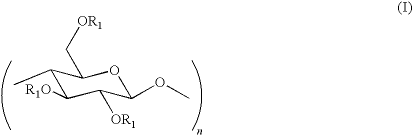

wherein n is an integer sufficient to produce a polymer with a weight-average molecular weight (Mw) in the range of about 70,000 to 3,000,000;

R₁ is, independently at each occurrence, H or —CH₂CH₂O—R₂, provided that at least one R₁ will be —CH₂CH₂O—R₂; and

R₂ is, independently at each occurrence, H, R₃N+(R₄)₃, or R₃N+(R₄)R₅, provided that at least one R₂ will be R₃N+(R₄)₃ or R₃N+(R₄)R₅, wherein:
R₃ is C₁₋₆ alkylene or O—C₁₋₆ alkylene;
R₄ is, independently at each occurrence, C₁₋₆ alkyl; and
R₅ is a 4 to 5 member alkylene, alkenylene, heteroalkylene, or heteroalkenylene, such that, along with the nitrogen to which it is attached, R₅ forms a saturated or unsaturated 5 or 6 member ring.

The term “alkylene” refers to a diradical alkyl group. Unless specified otherwise, all radicals include optionally substituted embodiments. “Optionally substituted” refers to hydroxyl, alkoxy, carboxy, nitro, amino, amido, halo, or C₁₋₃ alkyl. Accordingly, for example, Formula I specifically contemplates R₃ C₁₋₆ alkylene as —CH₂CH(OH)CH₂— and —CH₂CH(OH)—. The R₃ portion of Formula I is generally considered a bridge or tether to connect the remainder of the quaternary ammonium (N+(R₄)₃ or N+(R₄)R₅) to the ethoxy portion (CH₂CH₂O—) of the cellulose ether. Examples of R₃ when it is O—C₁₋₆ alkylene include —O—CH₂—, —O—CH₂CH₂—, and —O—CH₂—CH(CH₃)—.

In a preferred embodiment, R₂ is R₃N⁺(R₄)₃, and R₃ is —CH₂CH₂— or —CH₂CH(OH)CH₂—. Preferably in this embodiment, R₄ is, independently, CH₃ or CH₂CH₃, and most preferably R₃ is —CH₂CH(OH)CH₂— and R₄ is CH₃ at all occurrences.

Quaternary ammonium cations are permanently charged, independent of the pH of their solution, unlike primary, secondary, or tertiary amines.

In one embodiment, the cationic degree of substitution (often referred to as the CS or cationic substitution) of the cationic hydroxyethyl cellulose is in a range from about 0.075 to about 0.8, preferably, about 0.15 to about 0.60. A range of about 0.15 to about 0.60 corresponds to a Kjeldahl nitrogen content of about 0.8% to about 2.5%. More preferably, the cationic hydroxyethyl cellulose has a Kjeldahl nitrogen content between 1.5 and 2.2%, which corresponds to a CS of about 0.3 to about 0.5.

In one embodiment, the cationic hydroxyethylcellulose has a Brookfield LVT determined solution viscosity of from about 5 cP (=mPa·s) to about 10,000 cP, preferably from about 5 cP to about 3,000 cP, measured as a one weight percent aqueous solution at 25° C.

In one embodiment, the cationic hydroxyethylcellulose has a Brookfield LVT determined solution viscosity of from about 10 cP to about 50 cP, measured as a one weight percent aqueous solution at 25° C.

In another embodiment, the cationic hydroxyethylcellulose has a Brookfield solution viscosity of from about 1250 cP to about 2250 cP, measured as a one weight percent aqueous solution at 25° C.

Molecular weight can be conventionally determined using size-exclusion chromatography, preferably using low angle laser light scattering, and preferably determined as weight-average molecular weight (Mw). In one preferred embodiment, the cationic hydroxyethylcellulose has a Mw of about 350,000 to 550,000 Daltons. In another embodiment, the cationic hydroxyethylcellulose has a Mw of about 560,000 to about 790,000 Daltons. In another preferred embodiment, the cationic hydroxyethylcellulose has a Mw of about 800,000 to about 2,000,000 Daltons.

Methods of making compositions of Formula I are known, for example, U.S. Pat. No. 3,472,840 discloses quaternary nitrogen-containing cellulose ethers having a degree of polymerization (number of anhydroglucose repeat units) of 50 to 20,000, preferably 200 to 5,000, but does not suggest either ingesting the quaternary nitrogen-containing cellulose ethers nor their use in treating metabolic syndrome.

WO 2001/048021 discloses highly charged cationic cellulose ethers which are substituted with at least about 3.0 wt. % cationic substituent (based on Kjeldahl nitrogen measurements). The conversion of Kjeldahl nitrogen to CS will depend on the ethylene oxide (EO) substitution of the HEC, but at an EO MS of 2.0, 3.0% nitrogen corresponds to a cationic substitution (CS) of 0.79. For HEC with an EO MS of 1.0, 3.0% Kjeldahl nitrogen corresponds to a CS of 0.65.

Cationic HEC is available under the tradename UCARE™ from The Dow Chemical Company, and has an CTFA (Cosmetic, Toiletry, and Fragrance Association) designation of Polyquaternium-10. Cellulose ethers which comprise 1.5-2.2 weight percent of cationic nitrogen are sold commercially by the Amerchol division of The Dow Chemical Company under the trademark UCARE™ Polymers JR. Cellulose ethers which comprise 0.8-1.1 weight percent of cationic nitrogen are sold commercially by the Amerchol division of The Dow Chemical Company under the trademark UCARE™ Polymers LR.

In practice, cationic hydroxyethylcelluloses (c-HEC) may be formed by treating hydroxyethylcellulose with quaternary ammonium alkylating agent, for example, 3-chloro-2-hydroxypropyltrimethylammonium chloride or glycidyltrimethylammonium chloride. However, one change from conventional practice is that any steps of surface treating the c-HEC particles, most commonly cross-linking with glyoxal, to prevent undesirable dispersion and hydration problems (characterized by lumping) should be omitted. In one embodiment, the cationic hydroxyethylcellulose is substantially free of glyoxal.

The cationic hydroxyethylcellulose which are most useful in the present invention are water-soluble. The term “water-soluble” as used herein means that the cationic hydroxyethylcellulose has a solubility in water of at least 2 grams, preferably at least 3 grams, more preferably at least 5 grams in 100 grams of distilled water at 25° C. and 1 atmosphere.

In one embodiment, the oral ingestion form is a medicament or pharmaceutical containing cationic hydroxyethylcellulose. In a preferred embodiment, the CS of the cationic hydroxyethylcellulose is less than 0.6.

In one embodiment, the oral ingestion form is a food or beverage containing cationic hydroxyethylcellulose.

In one embodiment, the oral ingestion form is a nutraceutical or dietary supplement containing cationic hydroxyethylcellulose.

The cationic hydroxyethylcellulose can be administered or consumed in a number of conventional methods. Those skilled in the art readily understand the various means of formulating oral ingestion forms. In one embodiment, the oral ingestion form contains from about 0.25 g to about 4 g of cationic hydroxyethylcellulose.

In one embodiment, the present invention provides a method of preventing or treating a metabolic disorder in an individual, comprising administering to the individual an effective amount of a water-soluble cationic hydroxyethylcellulose.

“Individual” refers to an animal, preferably a mammal, more preferably, a human. Alternatively, the individual may be a mammal in need of weight loss, for example conventional pets, including but not limited to, dogs, cats, and rodents, or agricultural animals, including but not limited to, horses, cattle, swine, and sheep. The present examples take advantage of several accepted animal models designed to replicate human responses to treatment. The gold standard for a diet induced obese (DIO) model is the male C57BL/6J mouse. The C57BL/6J mouse develops an obese phenotype only when allowed ad libitum access to a high-fat diet (typically, containing 40-60% of calories derived from fat (a control diet contains 5-10% fat)). The obesity in the C57BL/6J mouse results from both increase of adipocyte size and number of adipocyte cells. In addition to obesity, typical disorders developed by the C57BL/6J mouse on a high fat diet are gradually worsening insulin resistance, glucose intolerance, mild to modest hyperglycemia, dyslipidemia, hypoadiponectinemia, leptin resitance/hyperleptinemia and hypertension. The C57BL/6J mouse closely parallels the progression of common forms of the human Type II diabetes in changes in central adiposity, the gradual course of development of diabetes, as well as the interaction of nutritional components and genetic variables.

The high fat fed hamster model lipoprotein system has lipoprotein cholesterol distribution, primary bile acids, cholesterol ester transfer protein activity and LDL receptor regulation very similar to that reported for the human lipoprotein system. Further, the distribution of phospholipid classes is more similar to humans than to rats. High fat fed hamsters are therefore favored models for dyslipidemias and cholesterol metabolism. High fat fed hamsters steadily increase body weight throughout their lifespan and thus generally show a reduction of weight gain as opposed to weight loss.

An effective amount of a water-soluble cationic hydroxyethylcellulose refers to the amount necessary to delay the development of a symptom in time or severity, or reduce the severity of a developing or developed symptom, or influence known biomarkers associated with the symptom.

Examples of symptoms of metabolic disorders include obesity, including abdominal adiposity, hepatic steatosis, atherogenic dyslipidemia, hypertension, insulin resistance with or without glucose intolerance, proinflammatory state, and prothrombotic state. As used herein, “preventing” refers to delaying the development of a symptom in time or severity, and “treating” or “ameliorating,” refer to reducing the severity of a developing or developed symptom.

Examples of biomarkers of metabolic disorders that are influenced include the expression or concentration of VLDL-C, LDL-C, HDL-C, adiponectin, leptin, fasting plasma glucose, fasting plasma insulin, liver lipids, liver glycerides, or free cholesterol esters in livers. It is understood that influencing includes both direct regulation of expression or indirect influence on expression, for example, by influencing the conditions or metabolites in a body tissue which lead to an altered gene expression or protein level. The level of expression or concentration could be determined after the intake of a cationic hydroxyethylcellulose by an individual, as compared to the level of expression or concentration after the intake of a non-effective material such as unmodified cellulose itself.

In one embodiment, the present invention provides a method of preventing or treating obesity and overweightness, comprising administering to the individual an effective amount of a water-soluble cationic hydroxyethylcellulose. In one embodiment, the individual experiences weight loss. “Weight loss” is defined as a reduction in an individual's body weight, preferably from reduced adipose tissue size or reduced adipocyte size and numbers. In one embodiment, the individual experiences a reduction in weight gain. “Reduction in weight gain” refers preferably to maintenance of an individual's body weight while eating a caloric excess.

In another embodiment, the present invention provides a method of preventing or treating atherogenic dyslipidemia, which is manifested in routine lipoprotein analysis by raised triglycerides and low concentrations of HDL cholesterol, comprising administering to the individual an effective amount of a water-soluble cationic hydroxyethylcellulose.

Adipocytes produce a variety of biologically active molecules, collectively known as adipocytokines or adipokines, including plasminogen activator inhibitor-1 (PAI-1), peroxisome proliferator-activated receptor alpha (PPARα), tumor necrosis factor alpha (TNFα), resistin, leptin, and adiponectin. In another embodiment of the present invention, the cationic hydroxyethylcellulose finds use in a method of influencing the level of expression or concentration of adiponectin, preferably raising expression or concentration of adiponectin, comprising administering to the individual an effective amount of a water-soluble cationic hydroxyethylcellulose. Adiponectin is a 30 kDa protein which is exclusively expressed in adipose tissues and is the most abundant circulating adipocytokines in both rodent and humans. Adiponectin has been found to be decreased in obesity, Type II diabetes, and coronary heart diseases. In obesity, adiponectin levels fall and leptin levels rise.

In another embodiment of the present invention, the cationic hydroxyethylcellulose finds use in a method of influencing the level of expression or concentration of leptin, preferably lowering expression or concentration of leptin, comprising administering to the individual an effective amount of a water-soluble cationic hydroxyethylcellulose. Leptin is also produced in adipocytes and is thought to play a key role in the regulation of body weight. In humans, leptin levels have been shown to be elevated with increasing adiposity in both men and women. A drop in adiponectin coupled with the loss of response to leptin, leads to ectopic lipid accumulation. When this occurs in muscle, it leads to insulin insensitivity.

In another embodiment of the present invention, a method of preventing or treating insulin resistance with or without glucose intolerance, comprising administering to the individual an effective amount of a water-soluble cationic hydroxyethylcellulose is provided. Insulin resistance is the condition in which normal amounts of insulin are inadequate to produce a normal insulin response from fat, muscle and liver cells.

Insulin resistance in fat cells results in hydrolysis of stored triglycerides, which elevates free fatty acids in the blood plasma. Insulin resistance in muscle reduces glucose uptake, whereas insulin resistance in liver reduces glucose storage, with both effects serving to elevate blood glucose. High plasma levels of insulin and glucose due to insulin resistance often lead to metabolic syndrome and Type II diabetes. In an insulin-resistant individual, normal levels of insulin do not trigger the signal for glucose absorption by muscle and adipose cells. To compensate for this, the pancreas in an insulin-resistant individual releases much more insulin such that the cells are adequately triggered to absorb glucose. On occasion, this can lead to a steep drop in blood sugar and a hypoglycemic reaction several hours after the meal.

Insulin resistance generally rises with increasing body fat content, yet a broad range of insulin sensitivity exists at any given level of body fat. Most people with obesity have postprandial hyperinsulinemia and relatively low insulin sensitivity, but variation in insulin sensitivity exists even within the obese population. A high plasma non-esterfied fatty acid (NEFA) level overloads muscle and liver with lipid, which enhances insulin resistance.

Measurements of insulin resistance, in a fasting state, include Homeostatic Model Assessment (HOMA), logarithm HOMA (log [HOMA]), and quantitative insulin sensitivity check index (QUICKI), which can be calculated as insulin resistance/sensitivity indices based on fasting glucose and insulin levels. These three indices employ fasting insulin and glucose levels to calculate insulin resistance, and each correlate reasonably with the results of clamping studies. In one embodiment, the individual experiences improved QUICKI index greater than about 20%, greater than about 25%, or preferably, greater than about 35%.

In another embodiment, the present invention provides a method of preventing or treating proinflammatory state, comprising administering to the individual an effective amount of a water-soluble cationic hydroxyethylcellulose. A proinflammatory state is recognized clinically by elevations of C-reactive protein (CRP).

In another embodiment, the present invention provides a method of preventing or treating a prothrombotic state, comprising administering to the individual an effective amount of a water-soluble cationic hydroxyethylcellulose. A prothrombotic state is characterized by increased plasma plasminogen activator inhibitor (PAI)-1 and fibrinogen.

In another embodiment, the present invention provides a method of preventing or treating metabolic syndrome, comprising administering to the individual an effective amount of a water-soluble cationic hydroxyethylcellulose. The term “metabolic syndrome” as used herein is characterized by at least three symptoms, more preferably four or more symptoms, selected from the group consisting of abdominal obesity, atherogenic dyslipidemia, hypertension, insulin resistance with or without glucose intolerance, proinflammatory state, and prothrombotic state. Symptoms or conditions associated with metabolic syndrome include, hyperglycemia, hyperinsulinaemia, hyperlipidaemia, impaired glucose metabolism, diabetic retinopathy, macular degeneration, cataracts, diabetic nephropathy, glomeruloscerosis, diabetic neuropathy, erectile dysfunction, premenstrual syndrome, vascular restenosis, and/or ulcerative colitis, angina pectoris, myocardial infarction, stroke, skin and/or connective tissue disorders, foot ulcerations, metabolic acidosis, arthritis, osteoporosis and conditions of impaired glucose tolerance, and cardiovascular diseases, or Type II diabetes to the extent that they are associated with the metabolic syndrome.

The desired time period of administering the cationic hydroxyethylcellulose can vary depending on the amount of cationic hydroxyethylcellulose consumed, the general health of the individual, the level of activity of the individual and related factors. Since metabolic syndrome or a symptom or condition associated with metabolic syndrome is typically induced by an imbalanced nutrition with a high fat content, it may be advisable to administer or consume the cationic hydroxyethylcellulose as long as nutrition with a high fat content is consumed. Generally administration of at least 1 to 12 weeks, preferably 3 to 8 weeks is recommended, or until symptoms are relieved.

It is to be understood that the duration and daily dosages of administration as disclosed herein are general ranges and may vary depending on various factors, such as the specific cellulose derivative, the weight, age and health condition of the individual, and the like. Preferably, the cationic hydroxyethylcellulose is administered or consumed in sufficient amounts throughout the day, rather than in a single dose or amount. The amount of administered cationic hydroxyethylcellulose is generally in the range of from 10 to 300 milligrams of cationic hydroxyethylcellulose per pound of mammal body weight per day. For a human, about 2 g to about 30 g, preferably about 3 g to about 15 g of cationic hydroxyethylcellulose are ingested daily.

In another embodiment, the present invention provides use of cationic hydroxyethylcellulose in the manufacture of a medicament for the treatment of metabolic disorders. In one embodiment, at least three symptoms selected from the group consisting of abdominal obesity, atherogenic dyslipidemia, hypertension, insulin resistance with or without glucose intolerance, proinflammatory state, and prothrombotic state are present. In one embodiment, the expression or concentration of VLDL, LDL, HDL, adiponectin, leptin, fasting plasma glucose, fasting plasma insulin, liver lipids, liver glycerides, or free cholesterol in liver is influenced. In one embodiment, the individual experiences weight loss. In one embodiment, the individual experiences a reduction in weight gain. In one embodiment, the individual experiences improved QUICKI index greater than about 20%, greater than about 25%, or preferably, greater than about 35%.

### EXAMPLES

The following examples are for illustrative purposes only and are not intended to limit the scope of the present invention.

### Wash Procedure:

Commercially available cationic hydroxyethylcellulose (c-HEC) is conventionally surface treated with glyoxal to prevent undesirable dispersion and hydration problems. This glyoxal must be removed before ingestion. The following examples use c-HEC that was washed substantially according to the following protocol to remove glyoxal.

A 12 liter, four-necked round-bottomed flask is fitted with a stirring paddle and motor, a nitrogen inlet, a pressure-equalizing addition funnel, and a reflux condenser connected to a mineral oil bubbler. The flask is charged with 1000 g of c-HEC, 6065 g of isopropyl alcohol, and 935 g of distilled water. The mixture is stirred for one hour under a steady flow of nitrogen to remove any entrained oxygen in the system. While continuing stirring under nitrogen, 100.0 g of 20% aqueous sodium hydroxide solution is added dropwise over 5 minutes, followed by stirring at ambient temperature for one hour under nitrogen. The slurry is neutralized by adding 42.5 g of glacial acetic acid, and after stirring for 15 minutes, the polymer is collected by vacuum filtration. The polymer is washed in a large Buchner funnel: four times with 10 liters of 4:1 acetone/water (by volume) and twice with 10 liters of pure acetone. The polymer is dried in vacuo at 50° C. overnight, yielding 938 g of off-white powder. The polymer has a volatiles content of about 4.9%, an ash content (as sodium acetate) of about 0.78%, and a glyoxal content of about 1.9 ppm.

### Example 1

An animal study was conducted with mature male Golden Syrian hamsters with a starting body weight of approximately 130 grams (LVG strain, Charles River, Wilmington, Mass.) in each of the diets specified below. The animal study was approved by the Animal Care and Use Committee, Western Regional Research Center, USDA, Albany, Calif.

The hamsters were divided into 28 groups, each group having approximately 8 hamsters. These groups were fed relatively high fat diets for a period of eight consecutive weeks, receiving doses of 1%, 2%, 4%, or 8% of cationic hydroxyethyl cellulose (c-HEC HV), a cationic hydroxyethyl cellulose having a relatively high viscosity and a CS of about 0.3 to about 0.5, or dietary fibers selected from hydroxypropyl methylcellulose (HPMC), (β-glucan, pectin, psyllium, xylan, or microcrystalline cellulose (MCC). 1000 g diet contained 20% fat (140 g of butter fat, 50 g corn oil, 10 g fish oil, and 1 g cholesterol), 20% protein (200 g casein), 468 g corn starch, 3 g DL-methionine, 3 g choline bitartrate, 35 g mineral mix, 10 g vitamin mix, and either 10 g (1%), 20 g (2%), 40 g (4%), or 80 g (8%) of c-HEC HV, HPMC, β-glucan, pectin, psyllium, xylan, or MCC. Fiber content was kept constant at 8% by weight by addition of an appropriate amount of MCC. MCC is generally regarded as the control group in these type of studies.

Each hamster body weight was recorded weekly and the amount of weight gain calculated as the difference from the initial body weight measurement. A portion of the data is summarized in Table 1 as the percent difference from the MCC control at a given week.

TABLE 1

| | Week 2 | Week 4 | Week 8 |
|---|---|---|---|
| β-glucan 4% | 242.5 | 374.0 | 133.7 |
| β-glucan 8% | 193.4 | 244.7 | 602.5 |
| Xylan 4% | 248.5 | 681.1 | 251.4 |
| Xylan 8% | 195.1 | 273.2 | 499.7 |
| Psyllium 4% | 127.9 | 462.3 | 235.7 |
| Psyllium 8% | −71.9 | −5.7 | 166.8 |
| Pectin 4% | 49.2 | 415.4 | 79.6 |
| Pectin 8% | −44.3 | −37.7 | 192.1 |
| HPMC 4% | 10.5 | 254.6 | 178.9 |
| HPMC 8% | −236.9* | −114.9* | 8.4 |
| cHEC 4% | −264.8* | −128.3* | −224.5* |
| cHEC 8% | −623.8* | −587.7* | −604.2* |

*statistically significant (p < 0.05)

The mature hamsters fed conventional dietary fibers gained weight on the diet. In other words, no statistically significant (p<0.05) reduction was observed for β-glucan, pectin, psyllium, xylan, or MCC. Surprisingly, analysis of body weight in hamsters fed diets containing 4% and 8% c-HEC showed statistically significant reduction in body weight. Even 2% c-HEC diet hamsters gained less weight than the top-performing 8% conventional fiber fed hamsters in the last three weeks of the study. Moreover, reduction in body weight gain was not due to a decrease in food intake. Similar results were observed for calculated energy intake.

### Example 2

An animal study was conducted with male Golden Syrian hamsters with a starting body weight of approximately 70 grams (LVG strain, Charles River, Wilmington, Mass.). The animal study was approved by the Animal Care and Use Committee, Western Regional Research Center, USDA, Albany, Calif. The hamsters were divided into 4 groups, each group having approximately 10 hamsters. These groups were fed relatively high fat diets for a period of four consecutive weeks, receiving doses of 5% medium viscosity c-HEC (“c-HEC LV”—a cationic hydroxyethylcellulose having a of about 0.3 to about 0.5), 5% high viscosity c-HEC (“c-HEC HV” from Example 1), 5% K250M HMPC or 5% MCC. The 1000 g diet contained 20% fat (140 g of butter fat, 50 g corn oil, 10 g fish oil, and 1 g cholesterol), 20% protein (200 g casein), 498 g corn starch, 3 g DL-methionine, 3 g choline bitartrate, 35 g mineral mix, 10 g vitamin mix, and either 50 g of c-HEC LV, c-HEC HV, HPMC, or MCC. Results are summarized in Table 2 as the percent difference from the MCC control.

TABLE 2

| | HPMC | c-HEC | c-HEC |
|---|---|---|---|
| | (Comparative) | LV | HV |
| Reduction in body weight gain | 13.6 | 22.5* | 25.6* |
| Reduction in R-adipose | 15.7 | 22.6* | 17.6 |
| Reduction in Liver weight | 34.1* | 38.0* | 39.2* |
| Reduction in VLDL cholesterol | 69.4* | 58.15* | 59.82* |
| Reduction in LDL cholesterol | 58.19* | 51.01* | 44.69* |
| Reduction in HDL cholesterol | 19.4* | 16.77* | 12.5* |
| Increase in plasma Adiponectin | 30.3* | 23.2* | 37.5* |
| Reduction in plasma Leptin | 23.9 | 44.2* | 48.6* |
| Reduction in fasting plasma glucose | 13.5 | 21.3* | 19.2* |
| Reduction in fasting plasma insulin | −14.4 | 13.4 | 67.3* |
| Increase in QUICKI index | −4.8 | 19.5* | 39.5* |
| Reduction in liver lipids | 21.1* | 26.1* | 18.6* |
| Reduction in liver triglycerides | −0.5 | 25.7* | −3 |
| Reduction in free cholesterol in liver | 41.7* | 20.0* | 26.9* |
| Increase of Bile Acids in feces | 171.6* | 78.4 | 40.1 |
| Increase of Sterols in feces | 40.1* | 14.8 | 20.8 |
| Increase of monoacylglycerides in | 566.3* | 329.7* | 340.7* |
| feces | | | |
| Increase of diacylglycerides in feces | 30.9* | 33.4 | 31.9 |
| Increase of triacylglycerides in feces | 74.6* | 5.6 | 13.6 |

*statistically significant (p < 0.05)

Reduction in body weight gain refers to the fact that although all the hamsters gained weight (as they had not yet reached a mature age), the MCC hamsters grew the heaviest. In addition to body weight, adipose tissue weight was evaluated to determine if total weight gain came from abdominal obesity. A significant reduction (p<0.05) was observed for c-HEC LV compared to the mean retroperitoneal adipose weight to MCC diet control. No significant differences were observed for both mesenteric adipose weight and kidney compared to the MCC diet control. c-HEC LV, c-HEC HV, and HPMC diets showed significant reductions (p<0.05) in mean liver weight compared to the MCC diet control.

The results are an indication that c-HEC HV, c-HEC LV, and HPMC cellulose derivatives are useful for preventing or treating atherogenic dyslipidemia in an individual, showing significant reductions (p<0.05) in mean VLDL-C, LDL-C, and HDL-C levels compared to the MCC diet control.

Adiponectin is involved in mediating lipid and glucose homeostasis, which correlates with other risk factors of metabolic syndrome. The results showed significant increases (p<0.05) in plasma adiponectin levels for c-HEC HV, c-HEC LV, and HPMC compared to the MCC control diet. These results suggest that these may have the ability to reduce insulin resistance and perhaps restore insulin sensitivity, possibly through regulating the expression of adipocytokines.

A significant reduction (p<0.05) in plasma leptin levels was observed for c-HEC HV and c-HEC LV when compared to MCC diet control. There does appear to be a good correlation of leptin levels and body weight and thus a strong link and correlation with obesity.

A significant decrease (p<0.05) in fasting-plasma glucose levels were observed for c-HEC HV and c-HEC LV. In addition, a significant reduction (p<0.05) in fasting-plasma insulin levels were observed for c-HEC HV which showed a 67% reduction compared to the MCC control diet. Collectively, the fasting-plasma glucose and insulin levels for the different cellulosic supplemented diets were further assessed by insulin resistance QUICKI indices. These surprising results provide evidence that c-HEC HV and c-HEC LV may help prevent or reduce the on-set of Type II diabetes, insulin resistance, obesity, and cardiovascular disease.

c-HEC HV, c-HEC LV, and HPMC supplemented diets showed statistically significant reductions (p<0.05) in mean total lipids compared to the control diet MCC. c-HEC LV exhibited decreases in plasma triglyceride levels of ˜28%. For liver free cholesterol, c-HEC HV, c-HEC LV, and HPMC showed as statistically significant reductions (p<0.05) in mean free cholesterol from the control diet MCC. Similarly, all of the diets showed statistically significant reductions (p<0.05) in mean total cholesterol from the control diet MCC.

Surprisingly, the bile acids, sterols and triglycerides did not show any statistically significant increases with cHEC supplemented diets. These results are surprising because HPMC facilitates the excretion of bile acids as well as cholesterol-derived metabolites in the feces of hamsters. Interestingly, monoacylglycerides were shown to be significantly increased (p<0.05) with the presence of cHEC LV or c-HEC HV. These unexpected results suggest that cHEC may have a different mechanism or mode-of-action compared to other fibers including HPMC.

### Protocols

All hamster body weights were recorded at the beginning and the end of the trial. Hamsters were fasted 12 hours before sacrificing and feces was collected from all the animals in the study, freeze dried and stored for lipid analysis. Plasma was prepared from cardiac blood collected into potassium EDTA. Livers were frozen and stored at −80° C.

Outlier Analysis: Multivariate analysis for the correlation of each biomarker was performed prior to outlier analysis. Outliers were determined based on Mahalanobis Distance. For most of the plasma protein biomarkers measured by ELISA or activity assays, this process could detect the outliers/variability caused by biological variability as well as differences that occurred during sample collection. Measurement variability was determined by % CV of the sample duplicate in each analysis. Outlier analysis of plasma lipid biomarkers and liver lipid biomarkers were analyzed in a similar fashion but separately from other biomarkers since sample handling and measuring differed. Outliers were omitted from the ANOVA analysis and means testing.

Simultaneous determination of cholesterol lipoproteins, based upon their particle size was performed by size-exclusion chromatography. An Agilent 1100 HPLC system was employed with a post-column derivatization reactor, consisting of a mixing coil (1615-50 Bodman, Aston, Pa.) in a temperature-controlled water jacket (Aura Industrials, Staten, N.Y.), and a Hewlett-Packard (Agilent, Palo Alto, Calif.) HPLC pump 79851-A was used to deliver cholesterol reagent (Roche Diagnostics, Indianapolis, Ind.) at a flow rate of 0.2 mL/min. Bovine cholesterol lipoprotein standards (Sigma Aldrich) were used to calibrate the UV detector using standard peak areas. Approximately 15μL of plasma was injected via an Agilent 1100 autosampler onto a Superose 6HR HPLC column (Pharmacia LKB Biotechnology, Piscataway, N.J.). The lipoproteins were eluted with a buffer containing 0.15 M NaCl, pH 7.0, 0.02% sodium azide at a flow rate of 0.5 mL/min.

Following sacrificing the lipoprotein levels were measured. The data was analyzed using JMP statistical software. Within each group the levels of species of interest were analyzed with JMP using One Way Analysis of Variance (ANOVA) and the means tested using the Tukey-Kramer HSD (Honestly Significant Difference) test.

Plasma samples were assayed for adiponectin based on a double-antibody sandwich enzyme immunoassay technique. Samples were diluted prior to the start of the assay with reagent buffers from the Adiponectin ELISA Kit (B-Bridge International, Inc. Mountain View, Calif.). After reconstituting all reagents, 100 μL of serially diluted adiponectin standards and diluted plasma sample were added to the appropriate number of antibody-coated wells. The plates were incubated at 22-28° C. for 60 minutes. Following incubation plates were washed three times with the wash buffer and 100 μL of biotinylated secondary anti-adiponectin polyclonal antibody was added to each well and allowed to incubate at 22-28° C. for 60 minutes. After washing three times with the wash buffer, a conjugate of horseradish peroxidase (HRP) and streptavidin was added to each well and allowed to incubate at 22-28° C. for 60 minutes. After washing the colorimetric substrate for the enzyme was added to all wells and incubated. The color development was terminated by the addition of a stop solution and the absorbance of each well was measured at 450 nm with a Synergy™ HT Multi-Detection Microplate Reader.

Plasma samples were assayed for leptin utilizing the Assay Design Leptin ELISA kit (Assay Designs, Inc., Ann Arbor, Mich.). All reagents were reconstituted according to the provided protocol. 100 μL of serially diluted leptin standards and plasma sample were added to the appropriate number of antibody-coated wells. The plate was sealed and incubated at 37° C. for

1 hour after brief mixing. After washing 7 times, 100 μL of the prepared Labeled Antibody solution was added to each well and incubated at 37° C. for 30 min. After washing nine times, 100 μL of the TMB Solution was added to each well, and the plates incubated 25° C. for 30 minutes in the dark. The reaction was terminated by adding 100 μL of stop solution to each well. The absorbance of each well was measured at 450 nm with a Synergy™ HT Multi-Detection Microplate Reader.

Plasma insulin levels were measured with a Mercodia Ultrasensitive Rat Insulin ELISA, which was run according to the manufacturers protocol. 50 μL of serially diluted calibrator and plasma sample were added to the appropriate number of antibody-coated wells, and incubated, shaking at a fast speed (400 rpm) on an orbital shaker at room temperature (25° C.) for 2 hours. After washing six times, 250 μL of the TMB Chromagen Solution were added to each well and incubated, shaking at a fast speed (400 rpm) on an orbital shaker, at room temperature (25° C.) for 30 minutes. The reaction was terminated by adding 50 μL of stop solution to each well. The absorbance of each well was measured at 450 nm with a Synergy™ HT Multi-Detection Microplate Reader.

The concentration of glucose (mg/dL) in hamster plasma samples was determined using the Roche Diagnostics/Hitachi 914 Clinical Analyzer and Roche Diagnostics Glucose/HK Assay kit according to the manufacturer's instructions. The measuring range for this assay is 2-750 mg/dL with a detection limit of 2 mg/dL. The Gluco-quant Glucose/HK assay uses two reagent solutions: R1 is 100 mmol/L, pH 7.8 TRIS buffer, 4 mmol/L Mg ⁺², ≧1.7 mmol/L ATP, ≧1.0 mmol/L NADP, and a preservative; R2 is 30 mmol/L, pH 7.0 HEPES buffer, 4 mmol/L Mg ⁺², ≧8.3 U/mL hexokinase (yeast), ≧15 U/mL glucose-6-phosphate dehydrogenase (E. coli), and a preservative. For calibration, the C.F.A.A. (Calibrator for Automated Systems) calibrator was used. For assay verification (quality check), Precinorm U Plus and Precipath U Plus control samples were analyzed. Plasma samples were thawed, loaded into sample cups, and analyzed as described above for the clinical analyzer assays.

The QUICKI index was calculated from fasting plasma glucose (mmol/L) and plasma insulin (mU/L) concentrations as follows:

QUICKI−1/(log [glucose]+log [insulin])

Analysis of bile acids, sterols, mono-, di-, and tri-glycerides from fecal samples. A lyophilized, ground feces sample (0.15 g+/−0.05 g) was weighed and mixed with 3.5 g of sand in a Dionex ASE extraction cell. A 100 μL aliquot of internal standard spiking solution (500 μg/mL in THF) was added to each sample (50 μg IS). The cell was placed in a Dionex Accelerated Solvent Extraction (ASE) system, and the extraction was carried out with 60/40 hexane/2-propanol with 2% acetic acid at 60° C. and 2175 psi (static 10 min). The extract (20 mL) was collected in a pre-weighed vial and shaken and 2 mL transferred to a separate vial for determination of cholesterol and triglycerides (see below). The remaining 18 mL of extract was evaporated to dryness under a stream of nitrogen (65° C., 45 min, 8 psi). 8 mL of acetonitrile was added to the vial and again evaporated to dryness and constant weight under a stream of nitrogen (45° C., 45 min, 10 psi). The residue was weighed to determine % total lipids. The residue was reconstituted in 0.9 mL of 2/6 tetrahydrofuran/[50/50 mobile phases A/B], and the solution was filtered through a 10 mm, 0.2 um PTFE syringe filter into a 2-mL HPLC autosampler vial. The sample was analyzed by HPLC using the conditions outlined below:
Instrument: Agilent 1200RR HPLC system with Chemstation Rev B.02.01-SR1 (LC1200RR,1897 Building)
Column: Waters Acquity BEH C18 column, 2.1×100 mm, 1.7 μm; PN:186002352
Chemstation Method: Acquisition Lipids Sterols BA 4.M;
   Processing: Lipids Sterols BA IS_{—}50_{—}12-12-07 (date of calibration)
Column Temperature: 50° C.
Flow Rate: 0.250 mL/min
Injection Volume: 2 μL (with needle wash in flushport, 1 s)
Mobile Phase A: 53/23/24 MeOH/CAN/H2O with 30 mM ammonium acetate, plus 24 mL per L acetic acid
Mobile Phase B: 2-Propanol with 30 mM ammonium acetate

Gradient:

| Time (min) | % B |
|---|---|
| 0.01 | 4 |
| 6.0 | 36 |
| 8.0 | 48 |
| 17.0 | 51 |
| 18.0 | 73 |
| 31.0 | 85 |
| 34.0 | 96 |
| 35.0 | 4 |

Detection: ESA Biosciences, Corona Plus Charged Aerosol Detector (CAD)

### Example 3

Obese C57BL/6J(B6) male mice with a starting age of ·18 weeks were obtained from Jackson Laboratories (Seattle, Wash.). This animal study was conducted by Jackson Lab and adheres to the regulations outlined in the USDA Animal Welfare Act (9 CFR, Parts 1, 2, and 3) and the conditions specified in The Guide for Care and Use of Laboratory Animals (ILAR publication, 1996, National Academy Press).

The mice were divided randomly into groups of 10 animals. The mice were fed formulated diet D12492 (high fat diet, 60 kcal % fat) that were prepared by Research Diets Inc. (New Brunswick, N.J.) and blended with different doses that contained either METHOCEL K250M hydroxypropyl methylcellulose (“HPMC”) or c-HEC HV resulting in the weight reduction percent compared to high fat diet D12492 described in Table 3.

TABLE 3

| | | Day 8 | Day 12 | Day 15 |
|---|---|---|---|---|
| HPMC | 2% | −167.2 | −87.0 | −55.8 |
| (Comparative) | 4% | 89.3 | 65.5 | 78.0 |
| | 8% | 351.0* | 206.9* | 196.9* |
| c-HEC HV | 2% | 176.2 | 81.8 | 88.5 |
| | 4% | 499.2* | 275.7* | 255.4* |

*significant reduction (p < 0.05)

Analysis of body weight in these mice showed statistically significant lower body weight in mice fed diets comprising of c-HEC-3% (not in Table), c-HEC-4%, and HPMC-8% compared to the high-fat diet control. The lower concentration of c-HEC required to show significant weight loss suggests that c-HEC is surprisingly more efficacious for weight loss. Weight loss was not due to a decrease in food intake, as food consumption in mice in diets supplemented with c-HEC or HPMC was not significantly changed.

### Example 4

An animal study was conducted by Perry Scientific, Inc. (PSI; San Diego Calif.) and adheres to the regulations outlined in the USDA Animal Welfare Act (9 CFR, Parts 1, 2, and 3) and the conditions specified in The Guide for Care and Use of Laboratory Animals (ILAR publication, 1996, National Academy Press). The protocol was approved by PSI's Institutional Animal Care and Use Committee prior to initiation of the study. Perry Scientific, Inc. is an AAALAC accredited facility. The study was carried out in a typical mouse DIO model. Obese C57BL/6J(B6) male mice with a starting age of ˜18 weeks were obtained from Jackson Laboratories (Seattle, Wash.).

The mice were divided randomly by weight into 9 groups of 10 animals each. These groups were fed relatively high fat diets (PROLAB RMH 2500 rodent diet at 60 kcal % fat) for a period of four consecutive weeks, receiving doses by weight of 0.5%, 1%, 2%, or 4% high viscosity c-HEC (“c-HEC HV” described in from Example 1), 1%, 2%, 4% or 8% HPMC (METHOCEL K250M HPMC, available from The Dow Chemical Company), or control with no c-HEC or dietary fiber.

Some of the results are summarized in Table 4 as the percent difference from the high fat control.

TABLE 4

| | 4% HPMC | 8% HPMC | 4% c- |
|---|---|---|---|
| | (Comparative) | (Comparative) | HEC HV |
| Reduction in body | 158.0* | 213.4* | 221.2* |
| weight gain | | | |
| Reduction in Mesentric- | 32.4* | 44.4* | 55.4* |
| adipose | | | |
| Reduction in Leptin | 46.3* | 77.0* | 77.6* |
| Reduction in fasting plasma | 22.0* | 26.8* | 37.6* |
| glucose | | | |
| Reduction in fasting plasma | 30.8 | 56.1 | 68.3* |
| insulin | | | |
| Increase in QUICKI | 18.1 | 20.6 | 42.7* |
| index % | | | |

*statistically significant (p < 0.05)

Analysis of body weight in these mice showed statistically significant lower body weight in mice fed diets comprising of cHEC-HV-4%, HPMC-4%, and HPMC-8% compared to the high-fat diet control. cHEC-HV 4% fed diet weight loss is similar to HPMC 8% fed diet weight loss, suggesting that cHEC is more efficacious. Reduction in body weight gain for cHEC-4%, HPMC-4%, and HPMC-8% fed diets, was not due to a decrease in food intake as food consumption in mice in diets supplemented with cHEC or HPMC was not significantly changed Similar results were observed for calculated energy intake.

A significant reduction (p<0.05) was observed for cHEC-HV-4%, HPMC-4%, and HPMC-8% of 55.4%, 32.4%, and 44.4%, respectively, compared to the mean mesenteric adipose weight to high-fat diet control. These results correlated well with the changes in whole body weight. In the diet induced obese (DIO) model male C57BL/6J mice, obesity results from both adipocyte hypertrophy (increase of adipocyte size) and hyperplasia (increase of number of adipocyte cells), and the fat gained is deposited selectively in the mesentery. However, in hamsters the metabolism is different and fat is deposited in the retroperitoneal adipose tissue. Adiponectin levels were not shown to increase, but this is an expected anomaly of C57BL/6J(B6) mice previously observed with other DIO-mouse studies.

A significant decrease (p<0.05) in leptin levels was observed for cHEC 4%, HPMC 4%, and HPMC 8%. These results correlate well with the reductions in percent body weight and mesenteric adipose fat.

Surprisingly, a significant reduction (p<0.05) was only observed for cHEC-4% of 68.3%, in mean plasma insulin levels compared to the high-fat control diet. A significant reduction (p<0.05) in fasting-glucose levels was observed for cHEC-4%, HMPC-4%, and HPMC-8%, of 37.6, 22.0, and 26.8%, respectively, compared to the high-fat diet control mice, which were hyperglycemic. To further assess the effect of cellulosic supplemented diets on insulin resistance QUICKI index was evaluated. Surprisingly, a significant difference (p<0.05) was only observed for cHEC-4% of 42.7% compared to the high-fat diet control. These results were similar to the hamster study (example 1). To date cHEC is the only fiber that has been investigated that shows a significant reduction in both fasting plasma glucose and fasting-plasma insulin. This data on cHEC provides further support on the mechanism of this cellulosic fiber in improving insulin resistance for testing dietary or therapeutic reagents for the prevention or treatment of insulin resistance and Type II diabetes.

### Protocols

Animals were maintained on test for 4 weeks and were weighed prior to commencement of the study, once a week thereafter and at termination prior to blood draw. Food consumption was measured twice a week by comparing given versus residual diet. Mice were fasted 12 hours before sacrificing. Plasma glucose levels were analyzed at Perry Scientific. At sacrifice blood, was collected via cardiac puncture into anticoagulant, separated by centrifugation and shipped frozen for analysis of glucose, insulin, leptin and adiponectin levels. The mesenteric fat pad was removed and weighed at sacrifice.

Analysis of variance was used to examine the effect of treatment on plasma biomarkers, lipid levels, and body and tissue weights. Measurement variability was determined by % CV of the sample duplicate in each analysis. Means Comparison—to facilitate multiple comparisons among bigger number of diet groups, the Tukey-Kramer HSD (Honestly Significant Difference) test is normally used for this type of mean comparison, which holds the overall confidence level of 95%. Pearson correlation coefficient was determined to investigate relationship between different biomarkers and parameters. JMP® 7.0.2 (SAS Institute Inc., Cary, N.C.) was used for the statistical analysis.

Mouse EDTA plasma samples were assayed for adiponectin based on a double-antibody sandwich enzyme immunoassay technique. Samples were diluted with reagent buffers from the Adiponectin ELISA Kit, B-Bridge International, Inc. (Mountain View, Calif.). 100 μL of serially diluted adiponectin standards and diluted plasma samples were added to the appropriate number of antibody-coated wells. The plates were incubated at 22-28° C. for 60 minutes. The plates were washed three times with the wash buffer and 100 μL of biotinylated secondary anti-adiponectin polyclonal antibody was added to each well and allowed to incubate at 22-28° C. for 60 minutes. After washing three times with the wash buffer, a conjugate of horseradish peroxidase (HRP) and streptavidin was added to each well and allowed to incubate at 22-28° C. for 60 minutes. After washing, the colorimetric substrate for the enzyme is added to all wells and incubated. Color development was terminated by the addition of a stop solution and the absorbance of each well was measured at 450 nm with a Synergy™ HT Multi-Detection Microplate Reader.

Plasma samples were assayed for leptin utilizing the Murine Leptin Immunoassay kit (R&D Systems, Minneapolis, Minn.). All reagents, plasma samples were diluted with the Calibrator Diluents 20× prior to assay. 50 μL of Assay Diluent was added to each sample well followed by the addition of 50 μL of serially diluted leptin standards and diluted plasma sample to the appropriate number of antibody-coated wells. Plates were incubated at room temperature (˜25° C.) for 2 hours and washed five times. 100 μL of antibody-enzyme conjugate solution were added to each well, incubated at 25° C. (room temperature) for 2 hours and washed five times with the wash buffer. 100 μL of the TMB Chromagen Solution were added to each well. The color reaction was terminated by adding 100 μL of stop solution to each well and the absorbance of each well measured at 450 nm with a Synergy™ HT Multi-Detection Microplate Reader.

Insulin was measured using the Mercodia Ultrasensitive Mouse Insulin ELISA according to the manufacturers protocol. 50 μL of serially diluted calibrator and plasma sample were added to the appropriate number of antibody-coated wells and incubated in the wells, shaking at a fast speed (900 rpm) on an orbital shaker at ˜37° C. for 2 hours. After washing six times, 200 μL of the TMB Chromagen Solution were added to each well and incubated, shaking at a fast speed (400 rpm) on an orbital shaker, at room temperature (25° C.) for 30 minutes. The reaction was terminated by adding 50 μL of stop solution to each well. The absorbance of each well was measured at 450 nm with a Synergy™ HT Multi-Detection Microplate Reader.

The QUICKI index was calculated from fasting plasma glucose (mmol/L) and plasma insulin (mU/L) concentrations as follows:

QUICKI−1/(log [glucose]+log [insulin])

It is understood that the present invention is not limited to the embodiments specifically disclosed and exemplified herein. Various modifications of the invention will be apparent to those skilled in the art. Such changes and modifications may be made without departing from the scope of the appended claims.

Moreover, each recited range includes all combinations and subcombinations of ranges, as well as specific numerals contained therein. Additionally, the disclosures of each patent, patent application, and publication cited or described in this document are hereby incorporated herein by reference, in their entireties.

1. An oral ingestion form comprising cationic hydroxyethylcellulose.

2. The oral ingestion form of claim 1, wherein the oral ingestion form is a medicament, pharmaceutical, food, beverage, food additive, nutraceutical, or dietary supplement.

3. The oral ingestion form of claim 1, wherein the oral ingestion form is substantially free of glyoxal.

4. The oral ingestion form of claim 1, wherein the oral ingestion form is a dietary supplement that contains from about 0.25 g to about 4 g of cationic hydroxyethylcellulose.

5. The oral ingestion form of claim 1 or 5, wherein the oral ingestion form contains an effective amount of cationic hydroxyethylcellulose to ameliorate at least three symptoms of selected from the group consisting of abdominal obesity, atherogenic dyslipidemia, hypertension, insulin resistance with or without glucose intolerance, proinflammatory state and prothrombotic state.

6. The oral ingestion form of claim 1, wherein the cationic hydroxyethylcellulose has a CS of about 0.3 to about 0.5.

7. A method of preventing or treating a metabolic disorder in an individual, comprising:
administering to the individual an effective amount of a water-soluble cationic hydroxyethylcellulose.

8. The method of claim 7, wherein at least three symptoms selected from the group consisting of abdominal obesity, atherogenic dyslipidemia, hypertension, insulin resistance with or without glucose intolerance, proinflammatory state, and prothrombotic state are present.

9. The method of claim 7, wherein the expression or concentration of VLDL, LDL, HDL, adiponectin, leptin, fasting plasma glucose, fasting plasma insulin, liver lipids, liver glycerides, or free cholesterol in liver is influenced.

10. The method of claim 7, wherein the individual experiences weight loss.

11. The method of claim 7, wherein the individual experiences a reduction in weight gain.

12. The method of claim 7, wherein the individual experiences improved QUICKI index greater than about 20%, greater than about 25%, or preferably, greater than about 35%.
